(19) United States
(12) Patent Application Publication
(10) Pub. No.: US 2015/0275269 A1
(43) Pub. Date: Oct. 1, 2015

(54) METHOD FOR PURIFYING NUCLEIC ACID AND KIT

(71) Applicant: THE EMERTER COMPANY, Shanghai (CN)

(72) Inventors: Li Li, Pudong (CN); Yongmei Li, Pudong (CN); Yulei Fu, Pudong (CN); Liang Cheng, Pudong (CN)

(73) Assignee: THE EMERTHER COMPANY, PUDONG (CN)

(21) Appl. No.: 14/438,585

(22) PCT Filed: Oct. 25, 2013
(86) PCT No.: PCT/CN2013/085968

§ 371 Date: Apr. 24, 2015

(30) Foreign Application Priority Data
Oct. 26, 2012 (CN) 201210418994.X

(51) Int. Cl. C12Q 1/68

(52) U.S. Cl. CPC C12Q 1/6806

(57) ABSTRACT

The present invention discloses a method for purifying nucleic acids and a kit. In particular, the present invention discloses a reagent combination for purifying nucleic acids from a specimen containing nucleic acids, a kit made based on the reagent combination, and a method for purifying nucleic acids using the reagent combination or the kit.

### FIELD

This present invention relates to a method and a kit for rapid purification of nucleic acids from specimen containing nucleic acids. The method is used to quickly and conveniently extract nucleic acids, which is especially suitable for extraction of nucleic acid substances from a variety of samples, such as whole blood, cells and tissues, etc.

### BACKGROUND

As the carrier of genetic information, nucleic acids are the material basis of gene expression. In addition to their important role in normal growth, development, and reproduction of organisms, nucleic acids are closely related to unusual circumstances of the life, such as tumor development, radiation injury, and genetic diseases, etc. Therefore, extraction and purification of nucleic acids is an essential process in molecular biology and medical research.

According to separation principle, the existing separation techniques include phenol-chloroform method, salting out techniques, ion exchange and oxidized silica adsorption.

A common method for separating nucleic acids is “charge-switch” method. Its principle is to first bind negatively charged nucleic acids to a positively charged nucleic acid binding phase at a pH below its pKa₁; and then the positively charged nucleic acid binding phase is neutralized at a pH above its pKa₂, causing the dissociation of nucleic acids from the nucleic acid binding phase. Some commercial kits use this technique for purification of genomic DNA from whole blood. However, this method requires proteinase K to digest sample in the process, which is not convenient for storage and usage of the kits.

Silica magnetic microparticles are also used for isolation of genomic DNA from whole blood. Such kits bind nucleic acids to the silica surface in a solution containing chaotropic salt, and then use low salt buffer and water to elute purified genomic DNA. However, DNA obtained using this method has low yield and low purity.

According to phase separation, current separation techniques include liquid phase and solid phase nucleic acid separation techniques.

The liquid nucleic acid separation techniques, such as the phenol-chloroform method, include processes such as sedimentation, centrifugation, etc., and often require complicated and time-consuming steps with liability of exposure to toxic reagents. As a result, DNA yield is low using this method and it is difficult to automate the process.

The solid phase nucleic acid purification methods bind nucleic acids to a solid phase carrier, and selectively wash off impurities. Such kits, e.g. silica spin column genomic DNA kit, have been widely used in the laboratory. Compared to the phenol-chloroform extraction method, the solid phase purification methods are simple and free of toxic and hazardous reagents. However, the solid phase purification methods still often requires centrifugation or filtration during the solid-liquid separation process, which can be cumbersome and is difficult to automate.

In summary, current nucleic acid separation techniques still have some deficiencies, including but limiting to the followings: use of protease digestion increases the total nucleic acid extraction time; kits need to be stored at specific temperature; cell lysis and binding of nucleic acids to a solid phase are separate processes, which increases purification steps; nucleic acids obtained after elution contain a high level of salt, which is unfavorable to subsequent molecular biology experiments; efficiency of nucleic acid extraction is low and purified nucleic acids contain relatively high protein and other impurities. Therefore, there is an urgent need to develop an efficient, fast, automated method to extract nucleic acids that are of high purity and low interference.

### SUMMARY OF THE INVENTION

The object of the present invention is to provide an efficient, fast, automated method, a reagent combination, and a kit to extract nucleic acids with high purity and low interference.

The first aspect of the present invention provides a reagent combination, which can be used for purification of nucleic acids from samples containing nucleic acids, wherein the reagent composition comprises:
1) a nucleic acid binding phase: the nucleic acid binding phase is used to adsorb and/or bind nucleic acids to be purified, and the nucleic acid binding phase has a surface with modification containing terminal carboxyl groups; and
2) a cell lysis-binding solution or corresponding components for preparation of the cell lysis-binding solution. The cell lysis-binding solution is used to lyse cell membrane, release nucleic acids to be purified, and enhance the binding of the nucleic acids and the aforementioned nucleic acid binding phase.

In another preferred embodiment, the composition of the cell lysis solution used in the cell lysis step and the DNA binding solution used in the binding step is the same or the cell lysis solution and the DNA binding solution is one solution (the cell lysis-binding solution).

In another preferred embodiment, the “terminal” could be the end of a straight-chain or a branched-chain.

In another preferred embodiment, the nucleic acid binding phase is silica magnetic microparticles or silica plates which are modified with terminal carboxyl groups.

In another preferred embodiment, the “same composition” refers to the components of the cell lysis solution and the DNA binding solution, other than the solvent (water or a mixed solvent of water and alcohol), that are 90% or more (preferably ≧95%, more preferably 100%) identical, and the concentrations of the respective components differ by ≦10% (preferably ≦5%, more preferably ≦2%).

In another preferred embodiment, the cell lysis-binding solution does not contain protease (e.g. proteinase K).

In another preferred embodiment, the cell lysis-binding solution completely or substantially prevents impurities (including protein, carbohydrates, RNA, etc.) from binding to the nucleic acid binding phase (bound impurity is ≦1% of the total impurities, more preferably ≦0.1% of the total impurities).

In another preferred embodiment, the nucleic acid binding phase is silica surface modified with the terminal carboxyl groups.

In another preferred embodiment, the nucleic acid binding phase is dextran surface modified with the terminal carboxyl groups.

In another preferred embodiment, the cell lysis-binding solution contains the following components:
(i) chaotropic salt, typically at a concentration of 3-6M;
(ii) alkali metal salt, typically at a concentration of 0.001-1M;
(iii) a surfactant, typically at a concentration of 2-5% (v/v);
(iv) a chelating agent, typically at a concentration of 0.5 mM-100 mM; and
(v) alcohol, typically at a concentration of 20-50% (v/v).

In another preferred embodiment, the chaotropic salt includes the salts of guanidine (e.g., guanidine hydrochloride).

In another preferred embodiment, the alkali metal salt comprises potassium chloride, sodium chloride, lithium chloride, or combinations thereof, preferably sodium chloride and/or lithium chloride.

In another preferred embodiment, the cell lysis-binding solution also contains (vi) other salts at a concentration of 0.01-3M. Wherein other salts comprise; monovalent salts, divalent salts, ammonium salts, or combinations thereof, preferably magnesium salt or zinc salt.

In another preferred embodiment, the cell lysis-binding solution contains the following components:
(i) chaotropic salt, at a concentration of 3-6M;
(ii) salt, at a concentration of 0.01-3M, wherein salt comprises monovalent salts (alkali metal salts and ammonium salts), divalent salts (salts such as magnesium salt, zinc salt), or combinations thereof;
(iii) a surfactant, at a concentration of 2-5% (v/v);
(iv) Optional chelating agent, at a concentration of 0-100 mM, preferably 0.5 mM-100 mM;
(v) alcohol, at a concentration of 20-50% (v/v); and
(vi) Optional other salt solution, at a concentration of 0.001-3M.

In another preferred embodiment, the surfactant includes Triton X-100, Tween20 or other non-ionic surfactants.

In another preferred embodiment, the chelating agents include EDTA, EGTA, CDTA, citrate, or combinations thereof.

In another preferred embodiment, the alcohol comprises ethanol or isopropanol.

In another preferred embodiment, the nucleic acid binding phase is silica magnetic microparticles or silica plates which are modified with terminal carboxyl groups.

In another preferred embodiment, the nucleic acid binding phase contains protonated groups, and the protonated group enhances the binding of nucleic acids to the nucleic acid binding phase; preferably, the protonated group is amine, including primary, secondary or tertiary amines.

In another preferred embodiment, the protonated group is:

R1-N—R2-(COOH)ₙ;

R1-NH—R2-(COOH)ₙ;

Wherein, R1 and R2 are independent alkyl groups with C₁-C₈ straight or branched chain, alkenyl groups with C₂-C₈ straight or branched chain, C₁-C₈ alkoxy (e.g., ethoxy) groups, C₆-C₁₅ aryl groups; and n=1˜3.

In another preferred embodiment, the alkyl, alkenyl, alkoxy, and aryl groups include substituted or unsubstituted groups. The substituted groups refer to one or more substituents selected from the following groups: halogen, C₁-C₄ alkyl groups, —OH, phenyl groups.

In another preferred embodiment, the lysis-binding solution contains alkali metal salts and the alkali metal salts are sodium salts, lithium salts, potassium salts, or combinations thereof.

In another preferred embodiment, the alkali metal salt is lithium chloride, sodium chloride or potassium chloride.

In another preferred embodiment, the alkali metal salt concentration is 0.1-3.0M, preferably 0.3-2.5M.

In another preferred embodiment, the lysis-binding solution may also contain other salts. The salt is not particularly restricted and can include (but not limited to) monovalent salts (alkali metal salts and ammonium salts), divalent salts (e.g., magnesium salts such as MgCl₂ and MgSO₄; zinc salts such as ZnCl₂ and ZnSO₄), or combinations thereof.

In another preferred embodiment, as the nucleic acid binding phase, the silica surface modified with terminal carboxyl groups can covalently attach to the following carriers: polymer materials, polysaccharide compounds, inorganic carriers, or combinations thereof.

In another preferred embodiment, the polymeric materials comprise polystyrene, polymethacrylate, cellulose, polyalcohol such as polyvinyl alcohol and polyvinyl butyral, and copolymers of these materials, or combinations thereof.

In another preferred embodiment, the polysaccharide compounds comprise dextran, agarose, cellulose, and derivatives of any of these materials, or combinations thereof.

In another preferred embodiment, the inorganic carriers comprise metal, glass, metal oxides and non-metallic oxides, carriers with a metal surface, magnetic microparticles, tubes, films, perforated plates, chips, microarrays, etc., or combinations thereof.

In another preferred embodiment, the reagent combination further comprises:
3) washing solution: wash to remove non-specific bound proteins, polysaccharides, lipids and other components.
4) Optional DNA elution solution: the elution solution provides an environment that is suitable for DNA elution, including water or a weak alkaline solution, such that the bound nucleic acids are able to dissociate from the nucleic acid binding phase.

Optionally, the reagent combination further comprises other common ingredients, such as buffer salts and chelating agents.

In another preferred embodiment, the buffer salts are Tris-HCl, phosphate buffer, HEPES, MOPS, or combinations thereof; the chelating agent is EDTA, EGTA, CDTA, citrate salts (e.g., sodium citrate) or combinations thereof.

In another preferred embodiment, the eluent is water or a biological buffer.

In another preferred embodiment, the pH value of the alkaline solution used for DNA elution is 7.5-9.

In another preferred embodiment, after a proper treatment for the cell wall, the nucleic acid extraction method can be applied to extract and purify nucleic acids from plant cells, bacteria, fungi, and virus samples.

The second aspect of the present invention provides a kit for purifying nucleic acids from samples containing nucleic acids. The kit comprises: (a) one or more containers, and one or more reagents selected from the reagent combination described in the first aspect of the present invention and contained in the containers, and (b) an instruction, describing the nucleic acid extraction method of the present invention. Preferably, the instruction also describes components of the cell lysis-binding solution, the binding and elution conditions, etc.

In another preferred embodiment, the kit of the present invention can also contain other optional common ingredients, including (but not limited to): washing reagents, buffers, and other cell wall lysis buffers, etc.

In the third aspect, the present invention provides applications of the reagent combination described in the first aspect or the kit described in the second aspect of the present invention. They are used to extract nucleic acids.

In the fourth aspect, the present invention provides a method to purify nucleic acids from a sample containing nucleic acids, comprising the steps of:

(a) Using the cell lysis-binding solution to treat the sample, causing lysis of cell membrane and release of nucleic acids to be purified, thereby obtain a crude mixture after the lysis process, and proteases are not added during the process;
(b) Mixing the crude mixture obtained after lysis treatment with the nucleic acid binding phase under appropriate binding conditions, so that the nucleic acid binding phase adsorbs and/or binds to nucleic acids to be purified, wherein the nucleic acid binding phase is the silica magnetic microparticles modified with terminal carboxyl groups;
(c) Separating the magnetic microparticles from the liquid phase of the mixture and obtain the magnetic microparticles bound with nucleic acids to be purified; and
(d) Washing off non-specifically bound impurities with a washing solution, and using a DNA elution solution to treat the magnetic microparticles bound with nucleic acids to be purified, so that the bound nucleic acids can be released from the nucleic acid binding phase and purified nucleic acids are then obtained.

In another preferred embodiment, the nucleic acid purification method comprises the use of the kit to extract nucleic acids as described in the second aspect of the present invention.

It should be understood that within the scope of the present invention, the technical features of the present invention and the technical features specifically described below (e.g. in the embodiments) can be combined to form new or preferred technical solutions. Due to space limitations, it is not repeated here.

### FIGURE DESCRIPTION

FIG. 1 shows the content of DNA purified from different volumes of mouse blood samples in the embodiment of Example 3.

FIG. 2 shows the electrophoresis profile of DNA purified from different volumes of blood samples in the embodiment of Example 3.

FIG. 3 shows the extraction results using the nucleic acid purification kit in the embodiment of Example 5.

### MODE OF CARRYING OUT THE INVENTION

Through long-term and in-depth research, the inventors unexpectedly found that the use of carboxylated silica magnetic microparticles as the nucleic acid binding phase can very efficiently, quickly, and easily extract nucleic acids, especially DNA. Specifically, experiments conducted by the inventors showed that in combination with the cell lysis-binding solution, the silica magnetic microparticles modified with terminal carboxyl groups can specifically bind to DNA molecules but do not or hardly bind to proteins, carbohydrates, RNA or the like impurities in the process of separation and purification of DNA, and so the purified DNA has higher purity. Based on this, the inventors completed the present invention.

### Magnetic Microparticles

As used herein, the terms “magnetic microparticles”, “magnetic microspheres” “magnetic beads”, and “magnetic particles” are used interchangeably, referring to the nucleic acid binding phase used in the present invention. In the present invention, the nucleic acid binding phase is a solid phase material used to bind nucleic acids.

In the present invention, there is no particular restriction on the size of magnetic microparticles. Generally, the particle size is 50 nanometers-2 micrometers, preferably 300-1000 nanometers.

In the present invention, there is no particular restriction on the materials of magnetic microparticles, which can contain or be made from a variety of magnetic materials. The magnetic microparticles can have a single layer structure or a multilayer structure (e.g., 2-6 layers).

In the present invention, as the nucleic acid binding phase, the magnetic microparticles are preferably modified magnetic microparticles, such as silica magnetic microparticles with surface modified with carboxyl groups or terminal carboxyl groups. A preferred nucleic acid binding phase is silica magnetic microparticles modified with terminal carboxyl groups.

A preferred nucleic acid binding phase can be paramagnetic microparticles or particles which can be separated by magnetic field.

Various magnetic microparticles (including modified magnetic microparticles), can be prepared by conventional methods in the art, or be obtained commercially. Representative examples include (but are not limited to): inorganic microparticles, bio-polymer microparticles, polymer microparticles.

### Cell Lysis-Binding Solution

The cell lysis solution is used to lyse cell membrane and release nucleic acids to be purified. The DNA binding solution is used to provide a low pH (e.g., pH5.5-7) condition, which is suitable for the binding of nucleic acids to the nucleic acid binding phase, and prevents impurities (including proteins, carbohydrates, RNA, etc.) from binding to the nucleic acid-binding phase.

In the present invention, the cell lysis solution and the DNA binding solution use the same or essentially the same composition, known as the cell lysis-binding solution. Thus, cell lysis and the binding of nucleic acids to the nucleic acid binding phase can use the same solution, or both can be performed in the same solution, achieving quick and easy extraction of nucleic acids.

The “same composition” refers to the components of the cell lysis solution and the DNA binding solution, other than the solvent (water or a mixed solvent of water and alcohol), that are 90% or more (preferably ≧95%, more preferably 100%) identical, and the concentrations of the respective components differ by ≦10% (preferably more preferably ≦2%).

In the present invention, the term “cell lysis-binding solution” refers to a solution that is not only used to lyse cell membrane to release nucleic acids to be purified, but also used to enhance the binding of nucleic acids to the nucleic acid binding phase.

In a preferred embodiment of the present invention, the cell lysis-binding solution contains the following components:
(i) chaotropic salt, typically at a concentration of 3-6M;
(ii) alkali metal salt, typically at a concentration of 0.001-1M;
(iii) a surfactant, typically at a concentration of 2-5% (v/v);
(iv) a chelating agent, typically at a concentration of 0.5 mM-100 mM; and
(v) alcohol, typically at a concentration of 20-50% (v/v).

In another preferred embodiment, chaotropic salt includes the salts of guanidine (e.g., guanidine hydrochloride).

In another preferred embodiment, the alkali metal salt comprises potassium chloride, sodium chloride, lithium chloride, or combinations thereof, preferably sodium chloride and/or lithium chloride.

In another preferred embodiment, the cell lysis-binding solution further contains (vi) other salts at a concentration of 0.01-3M, wherein the other salts comprise: monovalent salts, divalent salts, ammonium salts, or combinations thereof, preferably magnesium salts or zinc salts.

In another preferred embodiment of the present invention, the celllysis-binding solution contains the following components:
(i) chaotropic salt, at a concentration of 3-6M;
(ii) salt, at a concentration of 0.01-3M, wherein the salt comprises monovalent salts (alkali metal salts and ammonium salts), divalent salts (e.g., magnesium salts, zinc salts), or combinations thereof;
(iii) a surfactant, at a concentration of 2-5% (v/v);
(iv) Optional chelating agent at a concentration of 0-100 mM, preferably 0.5 mM-100 mM;
(v) alcohol, at a concentration of 20-50% (v/v); and
(vi) Optional other salt solutions, at a concentration of 0.001-3M.

In the present invention, the cell lysis-binding solution may or may not contain a chelating agent. There is no particular restriction on the chelating agent, which can include (but are not limited to): EDTA, EGTA, CDTA, citrate, or combinations thereof.

### Nucleic Acid Binding Phase

In the present invention, the nucleic acid binding phase is used to adsorb and/or bind to nucleic acids to be purified, and the nucleic acid binding phase has a surface with modification containing terminal carboxyl groups, preferably silica surface or dextran surface.

Preferably, the nucleic acid binding phase can be silica magnetic microparticles or silica plates modified with terminal carboxyl groups.

Magnetic microparticles with carboxyl groups on the surface is a common solid material used to extract and purify DNA. Under suitable DNA adsorption conditions (such as high molecular weight polyethylene glycol and highly concentrated sodium chloride), DNA can precipitate from aqueous solution and adsorbed to the carboxylated solid phase material.

Preferably, the nucleic acid binding phase contains protonated groups, and the protonated groups can contribute to binding of nucleic acids.

### Nucleic Acid Extraction Method

In the present invention, magnetic microparticles with surface specifically modified with carboxyl groups can be used to extract DNA (especially genomic DNA) through magnet separation. Typically, nucleic acids bind to the nucleic acid binding phase at a low pH; the elution of nucleic acids occurs at a pH greater than the binding pH.

With the method described in the present invention, cells are first lysed by the cell lysis-binding solution, then nucleic acids released from cells are specifically adsorbed onto the surface of the magnetic microparticles or microspheres described in the present invention, whereas proteins and other impurities are not adsorbed to the magnetic microparticles and remain in the solution. After that, the magnetic microparticles are separated from solution by magnet, and incubated with the elution solution to elute DNA of high purity.

In the method of the present invention, there is no need for centrifugation or use of protease (e.g., proteinase K) or a variety of other reagents, so the whole process is simple, particularly suitable for automation.

A preferred extraction method is to extract genomic DNA from cells.

The method of the present invention is applicable to biological materials (such as cells) without any particularly restriction. Representative examples include (but are not limited to): virus, chlamydia, bacteria, actinomycetes, yeasts, fungi, plant cells, and animal cells (e.g., mammalian animal cells) and the like. The invention is particularly applicable to extraction of nucleic acids from viruses, human and animals.

When the present invention is used for extraction of nucleic acids from a biological material containing cell wall, the biological material should be processed properly first (break cell wall).

Due to the high efficiency of the extraction process of the present invention, the method is not only suitable for extracting nucleic acids from a variety of samples, such as whole blood, cells and tissues, etc., but also particularly suitable for extracting genomic DNA from a small number of cells. In the present invention, a small number of cells generally refer to a single or 2-1000 cells (preferably 1-100 cells, more preferably 1-20 cells).

Nucleic acids which can be purified using the present invention may exist in the body fluid such as blood, urine, feces, saliva, sputum, or in tissues and organs. Nucleic acid samples can be obtained from carrier materials such as swab, smear specimens, or other fluid samples.

The cell lysis-binding solution in the present invention can be used to purify nucleic acids (including DNA). Representative examples include (but are not limited to) genomic DNA, cDNA, total RNA, etc.

In a preferred embodiment, the present invention uses the above-described nucleic acid binding phase and the reagent combination to extract nucleic acids. The present invention does not need a proteinase K digestion procedure and uses the same solution for cell lysis and binding of nucleic acids to the nucleic acid binding phase, thus enables quick, efficient and easy extraction of nucleic acids from various samples.

### A Reagent Combination and a Kit for Nucleic Acid Extraction

The present invention also provides a reagent combination and a kit used in combination with the method described in the present invention.

In the present invention, the reagent combination may comprise the nucleic acid binding phase described in the present invention and matching reagents which can be used with aforementioned nucleic acid binding phase. Typically, the reagent combination has one or more of the following reagents:

1) a nucleic acid binding phase: the nucleic acid-binding phase is a magnetic microparticle with a carboxyl group-modified silica surface, including magnetic microparticles with or without protonated modification.
2) a cell lysis solution: the cell lysis solution commonly comprises the following components: chaotropic salts, alkali metal salts, surfactants, chelating agents, alcohols (such as isopropanol).

In the present invention, since the cell lysis solution of the present invention (or the cell lysis-binding solution) contains the components specified above, there is no need to include proteinase K digestion procedure before, during or after cell lysis.

3) DNA binding buffer: The DNA binding buffer provides a suitable binding condition with low pH (e.g., pH5-7) so that nucleic acids bind to the nucleic acid binding phase but impurities (including proteins, carbohydrates, RNA, etc.) do not bind to the nucleic acid binding phase.

In a preferred embodiment, the cell lysis solution and the DNA binding buffer use the same or essentially the same composition, known as the cell lysis-binding solution. Thus, cell lysis and the binding between nucleic acids and the nucleic acid binding phase use the same solution, or are performed in the same solution, achieving quick and easy extraction of nucleic acids.

4) DNA elution solution: using water and a weak alkaline condition (e.g., pH7.5-8.5), bound nucleic acids can dissociate from the nucleic acid binding phase.

In the present invention, the kit comprises: (a) one or more containers, and one or more reagents selected from the reagent combinations described in the first aspect of the present invention and contained in the containers, and (b) an instruction, which describes the nucleic acid extracting method in the present invention. Preferably, the instruction also describes components of the cell lysis-binding solution and binding and elution conditions, etc.

It should be understood that the kit of the present invention can also contain any other common ingredients, including (but not limited to): washing reagents, buffers, and other cell wall lysis buffers, etc.

The Main Advantages of the Present Invention Include:

(1) Extraction process is quick and the whole operation is easy. With the present invention, cell lysis and specific binding of nucleic acids to the nucleic acid binding phase can be completed in one single step using the cell lysis-binding solution. Aqueous solution can be directly used to elute nucleic acids from the nucleic acid binding phase. No proteinase K treatment is needed in the process. The elution process is a simple wash step to release the bound nucleic acids from the nucleic acid binding phase. Therefore, the method only requires a few simple steps.
(2) The conditions used are mild. In the present invention, nucleic acids bind to the nucleic acid binding phase under mild conditions (pH5-7) and are eluted in water or a weak alkaline condition (pH7.5-8.5). The whole process is performed at room temperature.
(3) The yield of nucleic acids is high. One milligram of magnetic microparticles can bind up to 10 μg of genomic DNA, or extract more than 90% of human whole blood genomic DNA.
(4) The purity of extracted nucleic acids is high. Compared to other currently available kits and extraction methods, nucleic acids extracted using the nucleic acid purification kit descried in the present invention has high production, low impurities among other advantages. The extracted nucleic acids are suitable for a variety of downstream applications, such as PCR, nucleic acid hybridization, etc. No further purification is needed prior to the downstream applications.
(5) The whole process can be highly automated.

The below embodiments are used to further illustrate the invention. It should be understood that these embodiments are merely illustrative of the present invention and are not intended to limit the scope of the present invention. If specific conditions are not indicated in the following examples, they are usually in accordance with conventional conditions, or in accordance with the conditions recommended by the manufacturers. Unless otherwise indicated, percentages and parts are calculated by weight.

### Example 1

### Preparation of Magnetic Microparticles with Different Modification on the Surface

1) Preparation of amino silica magnetic microparticles: Weighed a certain amount of commercially available silanol silica magnetic microparticles, added anhydrous ethanol, water, concentrated aqueous ammonia and lastly 3′-aminopropyl triethoxysilane. The reaction mixture was stirred at room temperature for 3 hours and then the product was washed sequentially with anhydrous ethanol and distilled water to obtain magnetic microparticles with amino groups bound to the surface.
2) Preparation of carboxylated silica magnetic microparticles: Weighed a certain amount of commercially available silanol silica magnetic microparticles, added anhydrous ethanol, water, concentrated aqueous ammonia and finally 3′-glycidoxypropyl trimethoxy silane. The reaction mixture was stirred at room temperature for 3 hours. The product was then washed sequentially with ethanol and distilled water to obtain magnetic microparticles with epoxy groups bound to the surface. The magnetic microparticles with epoxy groups bound to the surface then reacted with 4-amino butyric acid to form silica magnetic microparticles with carboxylated surface.

### Example 2

### Use of the Kit for Purification of Nucleic Adds

Specific configuration examples of the nucleic acid purification kit described in the present invention include:

Magnetic microparticles: carboxylated silica magnetic microparticles made in-house as described above

The cell lysis-binding buffer: 4M guanidine hydrochloride, 2% Triton X-100, 0.1% SDS, 0.01% mercaptoethanol, 0.1M NaCl, 0.6M LiCl, 10 mM Tris-HCl (pH5.5), 1 mM EDTA, 25% isopropanol

Washing solution I: 200 mM NaCl solution, 0.8M LiCl, 70% ethanol, 50 mM Tris buffer (pH6.5.

Washing solution II; 70% ethanol

Elution solution: 1 mM EDTA, 10 mM Tris-HCl (pH8.0.

### Purification Steps:

1) Transferred 200 anticoagulanted blood to a 1.5 ml centrifuge tube, added 750 μl cell lysis-binding solution, shook the mixture well to lyse cells, let it stand for 5 min;
2) Added 1 mg magnetic microparticles, gently shook for 10 min;
3) Separated the magnetic microparticles by a magnetic separation rack and discard the supernatant;
4) Washed the magnetic microparticles twice using 850 μl washing solution I, gently shook for 2 min, separated the magnetic microparticles and discarded the supernatant;
5) Washed the magnetic microparticles twice using 850 μl washing solution II, gently shook for 2 min, separated the magnetic microparticles and discarded the supernatant;
6) Let the magnetic microparticles stand for 5 min to remove residual alcohol by evaporation;
7) Added 100 μL elution solution and mix well, let it stand at room temperature for 5 min;
8) Separated the magnetic microparticles by magnet, transferred the eluate to a centrifuge tube, which were used for DNA analysis and other downstream experiments.

### Example 3

### Comparison of Sever Surface Modified Magnetic Microparticles Used for Purification of Genomic DNA from Whole Blood

Samples: Mouse Blood Treated with Anti-Coagulant Heparin

### Materials:

Cell lysis-binding buffer: 4M guanidine hydrochloride, 2% Triton X-100, 0.1% SOS, 0.01% mercaptoethanol, 0.1M NaCl, 0.6M LiCl, 10 mM Tris-HCl (pH5.5), 1 mM EGTA, 25% isopropano.
Washing solution I: 100 mM NaCl solution, 0.8M LiCl, 70% ethanol, 50 mM Tris buffer (pH6.5)
Wash solution II: 70% ethanol
Elution solution: 1 mM EDTA, 10 mM Tris-HCl (pH8.0)

### Magnetic Microparticles:

Magnetic microparticles A: Commercially available silanol silica magnetic microparticles
Magnetic microparticles B: Aminated silica magnetic microparticles
Magnetic microparticles C; carboxylated silica magnetic microparticles

Using experimental procedure in Example 2 to purify genomic DNA, results are shown in the following table:

| | DNA con- | Total amount | | |
|---|---|---|---|---|
| Type of magnetic | centration | of DNA | A260/ | A260/ |
| microparticles | ng/μl | μg | A280 | A230 |
| Silanol silica | 20.5 | 2.05 | 1.56 | 0.34 |
| magnetic | | | | |
| microparticles | | | | |
| Aminated silica | 10.4 | 1.04 | 1.47 | 0.15 |
| magnetic | | | | |
| microparticles | | | | |
| Carboxylated silica | 81.3 | 8.13 | 1.88 | 1.50 |
| magnetic | | | | |
| microparticles | | | | |

The A260/A280 ratio of the extracted DNA was closer to 1.80 using the carboxylated silica magnetic microparticles, indicating the DNA extracted using the carboxylated silica magnetic microparticles had higher purity and less contamination from proteins and RNA. The A260/A230 ratio of the extracted DNA was about 1.50 using the carboxylated silica magnetic microparticles, which was higher than those obtained using the two other magnetic microparticles, indicating there were relatively less residual salt content in the DNA extracted using the carboxylated silica magnetic microparticles.

These results demonstrated that the total amount of extracted nucleic acids was significantly higher using carboxylated silica magnetic microparticles, than those using the silanol silica magnetic microparticles or the aminated silica magnetic microparticles.

### Example 4

### Purification of Genomic DNA from Mouse Blood Using the Nucleic Add Purification Kit

Samples: Mouse blood treated with anticoagulant heparin

### Materials:

Magnetic microparticles: silica magnetic microparticles modified with terminal carboxyl groups
Cell lysis-binding buffer: 4M guanidine hydrochloride, 2% Triton X-100, 0.1% SDS, 0.01% mercaptoethanol, 0.1M NaCl, 0.6M LiCl, 10 mM Tris-HCl (pH5.5), 1 mM CDTA, 25% isopropanol
Washing solution I: 100 mM NaCl solution, 0.8M LiCl, 70% ethanol, 50 mM Tris buffer (pH6.5)
Washing solution II: 70% ethanol
Elution solution: water

### Experimental Procedures:

1. Purification of Genomic DNA using the experimental procedures described in Example 2: Transferred 25, 50, 100, 200, 300, 400 μl of anticoagulanted blood to 1.5 ml centrifuge tubes and performed the following steps with each centrifuge tube; added 750 μl cell lysis-binding buffer and shook well to lyse cells, let them stand for 5 min; added 1 mg magnetic microparticles, gently shook for 10 min; adsorbed the magnetic microparticles by magnet and discarded the supernatant; washed magnetic microparticles twice using 850 μl washing solution I, gently shook for 2 min, adsorbed the magnetic microparticles by magnet and discarded the supernatant; washed magnetic microparticles twice using 850 μl washing solution II, gently shook for 2 min, adsorbed the magnetic microparticles by magnet and discarded the supernatant; let the magnetic microparticles stand for 5 min to remove residual alcohol by evaporation; added 1000 elution solution and mixed wed, let them stand at room temperature for 5 min; adsorbed magnetic microparticles by magnet, transferred the eluate to centrifuge tubes, which were tested for DNA content and purity and for electrophoresis analysis of nucleic acids.
2. PCR analysis of purified samples: PCR reaction system included 1 μl purified mouse genomic DNA, 2×PCR master buffer, 2 μl 3-glyceraldehyde phosphate dehydrogenase (GAPDH) upstream and downstream primers, and finally supplemented with sterilized ultrapure water to a final volume of 50 μl. Upstream and downstream primer sequences were 5′-AGAGTCCATGCCATCACTGCC-3′, 5′-GCCTGCTTCACCACCTTCTTG-3′, respectively. PCR reaction conditions: 94° C. pre-denaturation for 4 min, 94° C. denaturation for 30 s, 55° C. anneal for 30 s, 72° C. extension for 1 min, 30 cycles, 72° C. extension for 10 min. After the reaction, PCR products were used for electrophoresis analysis.

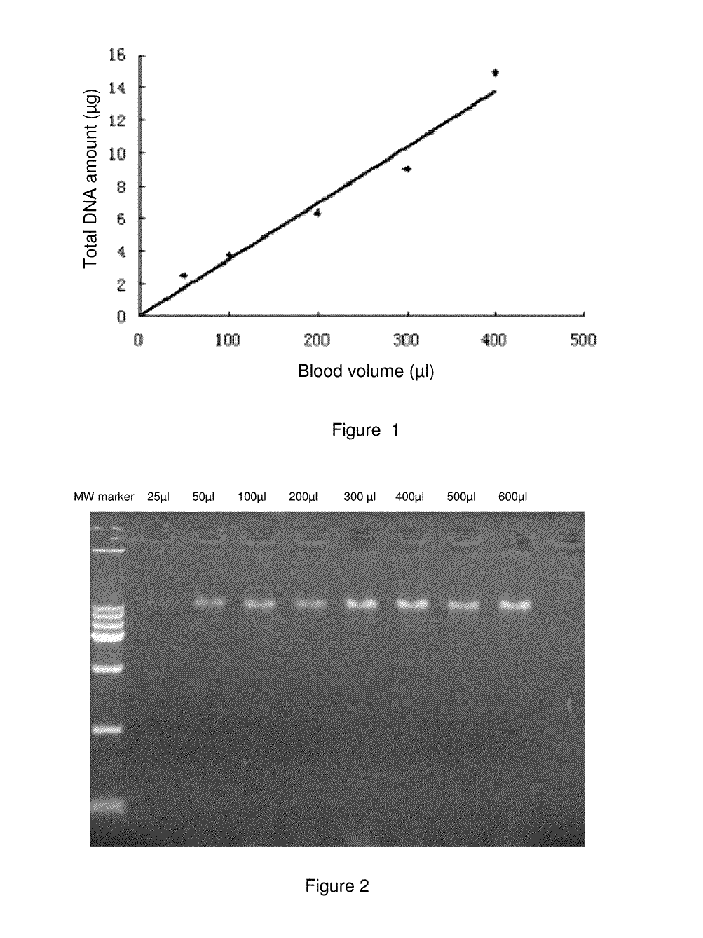

The results (FIG. 1) show that the kit described in the present invention was able to consistently extract DNA with high yield using different volumes of blood as the raw materials. Using the kit described in the present invention, 1 mg of magnetic microparticles could bind up to 15 μg of mouse genomic DNA.

### Examples 5

### Extraction of Human Genomic DNA Using the Nucleic Acid Purification Kit

### Materials:

Human whole blood samples treated with EDTA anticoagulant
Magnetic microparticles: silica magnetic microparticles modified with terminal carboxyl groups
Cell lysis-binding buffer: 4M guanidine hydrochloride, 2% Triton X-100, 0.1% SDS, 0.01% mercaptoethanol 0.1M NaCl, 0.6M LiCl, 10 mM Tris-HCl (pH5.5), 1 mM sodium citrate, 25% isopropanol
Washing solution I: 100 mM NaCl solution, 0.8M LiCl, 70% ethanol, 50 Tris buffer (pH6.5)
Wash solution II: 70% ethanol
Elution buffer: 1 mM EDTA, 10 mM Tris-HCl (pH8.0)
Theoretical gDNA value was calculated based on white blood cell count (each white cell contains 6.6 pgDNA).

### Purification Steps:

Purification of genomic DNA using the experimental procedures described in Example 2: Transferred 50, 100, 200, 300, 400 μl of anticoagulanted blood into 1.5 ml centrifuge tubes and perform the following steps in each centrifuge tube; added 750 μl cell lysis-binding solution and shook well to lyse cells, let them stand for 5 min; added 1 mg magnetic microparticles, gently shook for 10 min; adsorbed the magnetic microparticles by magnet and discarded the supernatant; washed the magnetic microparticles twice using 850 μl washing solution I, gently shook for 2 min, adsorbed the magnetic microparticles by magnet and discarded the supernatant; washed the magnetic microparticles twice using 850 μl washing solution II, gently shook for 2 min, adsorbed the magnetic microparticles by magnet and discarded the supernatant; let the magnetic microparticles stand for 5 min to remove residual alcohol by evaporation; added 100 μl elution solution and mixed well, let them stand at room temperature for 5 min; adsorbed magnetic microparticles by magnet, transferred the eluate to centrifuge tubes, which were used to measure DNA content and purity.

| Volume of | White blood | Theoretical | DNA content | DNA |
|---|---|---|---|---|
| blood | cell count | DNA content | after elution | recovery |
| (μl) | (×10³/μl) | (μg DNA) | (μg DNA) | (%) |
| 50 | 5.2 | 1.72 | 1.69 | 98.5 |
| 100 | 5.2 | 3.43 | 3.15 | 91.8 |
| 200 | 5.2 | 6.86 | 6.22 | 90.6 |
| 300 | 5.2 | 10.30 | 9.37 | 91.0 |

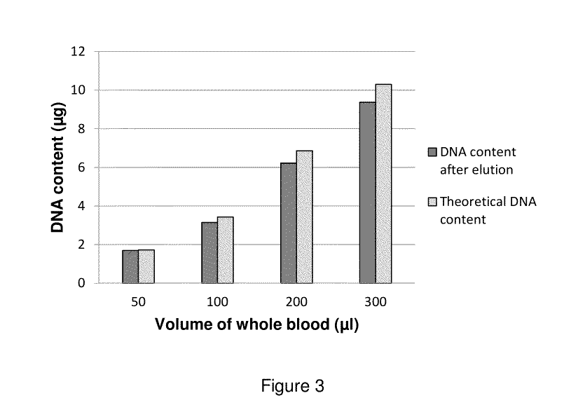

The extraction results using the nucleic acid purification kit are shown in FIG. 3, demonstrating that the kit of the present invention can extract more than 90% of the total genomic DNA.

### Example 6

### Preparation of Carboxylated Dextran Microparticles

Prepared 200 ml solution (concentration: 20 mg microparticles/ml solution) containing the commercially available dextran magnetic microparticles (particle size of about 40 μm), added sodium hydroxide (final concentration of 3M) and bromine hexanoic acid (final concentration of 20 mg/ml), stirred at room temperature for 3 hours. Dextran magnetic microparticles with carboxylated surface were obtained.

### Example 7

### Comparison of Purification of Genomic DNA from Whole Blood Using Dextran Magnetic Microparticles

Samples: mouse blood treated with anticoagulant heparin

### Materials:

Cell lysis-binding buffer: 4M guanidine hydrochloride, 2% Triton X-100, 0.1% SDS, 0.01% mercaptoethanol, 0.1M NaCl, 0.8M LiCl, 10 mM Tris-HCl (pH6.5), 45% isopropanol
Washing solution I: 100 mM NaCl solution, 0.8M LiCl, 70% ethanol, 50 mM Tris buffer (pH7.5)
Washing solution II: 70% ethanol
Elution solution: 1 mM EDTA, 10 mM Tris-HCl (pH8.0)

### Magnetic Microparticles:

Magnetic microparticles A: Commercially available dextran magnetic microparticles
Magnetic microparticles B: Carboxylated dextran magnetic microparticles prepared in Example 6

Using the experimental procedures for purification of genomic DNA described in Example 2, results are shown in the following table:

| | DNA con- | Total amount | | |
|---|---|---|---|---|
| Type of magnetic | centration | of DNA | A260/ | A260/ |
| microparticles | ng/μl | μg | A280 | A230 |
| Dextran magnetic | — | — | — | — |
| microparticles | | | | |
| Carboxylated | 27.3 | 2.73 | 1.92 | 1.15 |
| dextran magnetic | | | | |
| microparticles | | | | |

The A2603/280 ratio of the extracted DNA was closer to 1.80 using the carboxylated dextran magnetic microparticles, indicating DNA extracted in this way had higher purity and less contamination from protein and RNA. The A260/A230 ratio of the extracted DNA was about 1.50 using the carboxylated dextran magnetic microparticles, indicating relatively less residual salt content in the extracted DNA.

These results indicated that the total amount of extracted nucleic acids was significantly higher using carboxylated dextran magnetic microparticles, than that using the dextran magnetic microparticles.

All references disclosed herein are hereby incorporated by reference in their entirety as if each was incorporated individually. It should also be understood that, from the foregoing disclosure of the present invention, those skilled in the art may make various changes or modifications of the present invention, and all such changes or modifications are also intended to fail within the scope of the present invention.

1-12. (canceled)

13. A reagent combination for purifying nucleic acids from a sample containing nucleic acids, wherein the reagent combination comprises:
a) a nucleic acid binding phase for adsorbing and/or binding to the nucleic acids to be purified, wherein the nucleic acid binding phase has a surface with modification containing terminal carboxyl groups; and
b) a cell lysis-binding solution or corresponding components to prepare the cell lysis-binding solution to lyse cell membrane, release nucleic acids to be purified, and enhance the binding of nucleic acids to said nucleic acid binding phase.
wherein the cell lysis-binding solution is a single solution comprising the following components:
(i) chaotropic salt, at a concentration of 3-6M;
(ii) salt, at a concentration of 0.01-3M, wherein the salt comprises monovalent salts, such as alkali metal salts and ammonium salts, divalent salts, such as magnesium salts and zinc salts, or combinations thereof;
(iii) a surfactant, at a concentration of 2-5% (v/v); and
(iv) alcohol, at a concentration of 20-50% (v/v), wherein said alcohol is ethanol or isopropanol.

14. The reagent combination according to claim 13, characterized in that the nucleic acid binding phase is silica surface modified with terminal carboxyl groups.

15. The reagent combination according to claim 13, characterized in that the nucleic acid binding phase is dextran surface modified with terminal carboxyl groups.

16. The reagent combination according to claim 13, wherein the cell lysis-binding solution further comprises (v) chelating agent at a concentration of 0-100 mM, preferably 0.5 mM-100 mM.

17. The reagent combination according to claim 13, wherein the cell lysis-binding solution further comprises (vi) other salt solution, at a concentration of 0.001-3M.

18. The reagent combination according to claim 13, wherein the cell lysis-binding solution comprises the following components:
(i) chaotropic salt, at a concentration of 3-6M;
(ii) alkali metal salt, at a concentration of 0.001-3M;
(iii) a surfactant, at a concentration of 2-5% (v/v);
(iv) a chelating agent at a concentration of 0.5 mM-100 mM; and
(v) alcohol at a concentration of 20-50% (v/v), wherein said alcohol is ethanol or isopropanol.

19. The reagent combination according to claim 14, characterized in that the nucleic acid binding phase is silica magnetic microparticles or silica plates which are modified with terminal carboxyl groups.

20. The reagent combination according to claim 14, characterized in that, in the nucleic acid binding phase, the silica surface modified with terminal carboxyl functional groups can be covalently attached to the following carriers: polymer materials, polysaccharide compounds, inorganic carriers, or combinations thereof.

21. The reagent combination according to claim 13, characterized in that the reagent combination further comprises a washing solution to remove non-specifically bound proteins, polysaccharides, lipids, and other components.

22. The reagent combination according to claim 13, characterized in that the reagent combination further comprises DNA elution solution; wherein the elution solution includes water or a weak alkaline solution and provides a suitable environment for DNA elution, causing the bound nucleic acids to dissociate from the nucleic acid binding phase.

23. The reagent combination according to claim 13, characterized in that the nucleic acid binding phase contains protonated groups, and the protonated groups enhance the binding of nucleic acids to the nucleic acid binding phase.

24. The reagent combination according to claim 23, wherein the protonated group is amine, including primary, secondary or tertiary amines.

25. A kit used for purifying nucleic acids from a sample containing nucleic acids, characterized in that the kit comprises: (a) one or more containers, and one or more reagents selected from the reagent combination according to claim 13 and contained in the containers, and (b) an instruction, describing the nucleic acid extracting method.

26. A method for using the reagent combination according to claim 13, comprising the step of using the combination for extraction of nucleic acids.

27. A method for using the kit according to claim 25, comprising the step of using the kit for extraction of nucleic acids.

28. A method for purifying nucleic acids from a sample containing nucleic acids, characterized by comprising the steps of:
(a) Using a cell lysis-binding solution to treat the sample, causing cell lysis and release of nucleic acids to be purified; thereby obtaining a crude mixture after the lysis process;
and wherein the cell lysis-binding solution is a single solution;
(b) Mixing the crude mixture obtained after lysis treatment with a nucleic acid binding phase under suitable binding conditions, so that the nucleic acid binding phase adsorb and/or bind to nucleic acids to be purified, wherein the nucleic acid binding phase is silica magnetic microparticles modified with terminal carboxyl groups;
(c) Separating magnetic microparticles and the liquid phase of the mixture, thereby obtaining the magnetic microparticles bound with nucleic acids; and
(d) After using a washing solution to wash off non-specifically bound impurities, using a DNA elution solution to treat the magnetic microparticles bound with nucleic acids to be purified, so that the bound nucleic acids can be released from the nucleic acid binding phase and purified nucleic acids are then be obtained;
wherein no protease is added during the method and the method being performed at room temperature.

29. The method according to claim 28, wherein the cell lysis-binding solution comprises the following components:
(i) chaotropic salt, at a concentration of 3-6M;
(ii) salt, at a concentration of 0.01-3M, wherein the salt comprises monovalent salts, such as alkali metal salts and ammonium salts, divalent salts, such as magnesium salts and zinc salts, or combinations thereof;
(iii) a surfactant, at a concentration of 2-5% (v/v);
(iv) alcohol, at a concentration of 20-50% (v/v), wherein said alcohol is ethanol or isopropanol.

30. The method according to claim 28, wherein the cell lysis-binding solution further comprises the following component: (v) chelating agent at a concentration of 0-100 mM, preferably 0.5 mM-100 mM.

31. The method according to claim 28, wherein the cell lysis-binding solution further comprises the following component: (vi) other salt solution, at a concentration of 0.001-3M.

32. The method according to claim 28, wherein the cell lysis-binding solution comprises the following components:
(i) chaotropic salt, at a concentration of 3-6M;
(ii) alkali metal salt, at a concentration of 0.001-3M;
(iii) a surfactant, at a concentration of 2-5% (v/v);
(iv) a chelating agent at a concentration of 0.5 mM-100 mM; and
(v) alcohol at a concentration of 20-50% (v/v), and said alcohol is ethanol or isopropanol.
